(19) United States
(12) Patent Application Publication
(10) Pub. No.: US 2005/0021134 A1
(43) Pub. Date: Jan. 27, 2005

(54) Method of rendering a mechanical heart valve non-thrombogenic with an electrical device

(76) Inventor: John C. Opie, Scottsdale, AZ (US)

Correspondence Address: SQUIRE, SANDERS & DEMPSEY L.L.P.;Two Renaissance Square, Suite 2700, 40 North Central Avenue, Phoenix, AZ, 85004-4498, US

(21) Appl. No.: 10/883,574

(22) Filed: Jun. 30, 2004

Related U.S. Application Data
(60) Provisional application No. 60/484,038, filed on Jun. 30, 2003.

(51) Int. Cl. A61F 2/06

(52) U.S. Cl. 623/2.2; 623/1.24; 607/119

(57) ABSTRACT

A mechanical device for implantation into a patient's body is designed or modified to be electrically charged to prevent coagulation on the device, thereby extending the life of the device and alleviating the need for the patient to utilize anticoagulant therapy. The device may be a heart valve and is electrically charged by being connected to a power source. The power source is preferably a battery pack implanted in the body and is connected to the device by connector wires. The charge applied to the device may be negative or positive, as long as it helps to repel platelets and/or red blood cells from the device in order to help prevent coagulation on one or more surfaces of the device.

### RELATED APPLICATIONS

This application claims priority to U.S. Provisional Application Ser. No. 60/484,038, filed Jun. 30, 2003, to John C. Opie.

### FIELD OF THE INVENTION

The invention relates to medical devices permanently or semi-permanently implanted into the body and more particularly to a partially or totally non-thrombogenic mechanical device such as a heart valve.

### BACKGROUND OF THE INVENTION

Currently, patients who have an implanted mechanical device, particularly a mechanical heart valve, must usually be anti-coagulated (by taking anti-coagulation medication) for life due to the fact that the heart valve acts as a local initiator for coagulation. Among the known mechanical heart valve designs are those disclosed in U.S. Pat. Nos. 6,645,244, 6,395,024, 6,699,283, 6,638,303 and 6,582,464, and U.S. patent application Ser. Nos. 10/133,859 and 10/717,817, the respective disclosures of which are incorporated herein by reference.

Although not important for an understanding of the design or scope of the invention, a general medical description of the coagulation process and the body's down regulation of blood clotting is as follows. Once coagulation is initiated the internal and the external coagulation pathways converge into a common pathway at a point when Factor X is activated at the surface of the platelet. The intrinsic pathway begins when Factor XII is activated to XIIa by contact with a positively charged passive foreign surface. Co-factors in this activation conversion include prekallikrein, high molecular weight kininogen and Factor XI. These proteins form a surface-localized complex on the valve surfaces and will activate Factor XII. The activated Factor XIIa then converts Factor XI to XIa, and also converts prekallikreinin to its activated form, kallikrein, which in turn cleaves high molecular weight kininogen to bradykinin. Once Factor XIa is present, it cleaves plasminogen to form plasmin. Plasmin is the main protease involved with the fibrinolytic mechanism that restrains blood clotting. These processes activate Factor X at the plasma membrane of stimulated platelets but Xa may also occur on the vascular endothelium. Factor Xa production is the first step in the common pathway. It then activates Factor II (pro-thrombin) to generate the protease thrombin. Assembly of the plasma pro-thrombinase complex on the surface of activated platelets in the presence of Factor V, another co-factor, enhances the efficiency of pro-thrombin activation to thrombin on the platelet surface. Thrombin cleaves fibrinogen, which is a large asymmetric, soluble protein of 340-kilodaltons in three polypeptide chain pairs: alpha, beta and gamma. Thrombin first removes small peptides from the A chain of fibroinogen to form Fibrin I, which polymerizes end to end; further thrombin cleavage of small peptides from the B chain, leads to formation of Fibrin II molecules, which also polmerize side to side and are then cross linked via the gamma chain and subunits of plasma glutaminase (Factor XIII). An insoluble fibrin clot is the result.

Platelets that come into contact with foreign surfaces quickly interact with that surface. The initial reaction is for the surface of the platelet to grow irregular surface nodes or nobs. The nodes develop as alpha degranulation of the platelet occurs with associated thromboxane A2 release. That phenomenon is associated with alterations of the surface charge on the platelet, which become negatively charged with respect to the intracellular fluid of the platelet, which remains positively charged. Red blood cells undergo a similar activation process. These surface negative charges induce platelets to adhere to the foreign surface using an electrostatic initiation process thus commencing the intrinsic coagulation pathway, which ends with the formation of white thrombus. The platelet mesh soon entangles passing red blood cells and early red clot develops. The process extends and if a mechanical valve is left un-anti-coagulated, the valve will thrombose with disastrous results for the patient. Galvanization of intra-vascular materials has been studied previously. (Zimmermann M, Metz J, Ensinger W, Kubler W. Coronary Art Dis July 1995;6(7):581-6. Influence of surface texture and charge on biocompatibility of endovascular stents.) It has been determined that ion bombarded stents do not occlude by thrombus if the in-vitro surface potentials range between +120 mV and +180 mV, although these studies only lasted about four weeks. Alternatively, Godin C, Caprani A, remark in the Eur Biophys J, 1993;25(1):25-30—Interactions of erythrocytes with an artificial wall: influence of electrical charge, that an electrical charge on any biological surface plays a crucial role in its interaction with other molecules or surfaces. A maximal interaction of erythrocytes with the charged surface is calculated in the 0 to +10 microC/cm2 charge density and that a high positive surface charge (>10 microC/cm2) induces a progressive decrease in contact efficiency, which might be explained by a rearrangement of macromolecules on platelet or red blood cell surface or an effect of positively charged groups on the cell membrane. Whereas a negative surface charge produced a less efficient contact due to electrostatic repulsion forces.

Whereas the blood coagulation pathways involve a series of enzymatic activations of serine protease zymogens, down-regulation of blood clotting is influenced by a variety of natural anticoagulant mechanisms, including antithrombin III, protein C-protein S system and fibrinolysis. Healthy vascular endothelium promotes the activation of these down-regulation systems. In addition to the systems presented above, additional clotting down-regulation is managed with thrombomodulin formed from the endothelium it complexes with thrombin activated protein C—this relationship stimulates the release of tissue plasminogen activator (TPA). These factors acting in concert inactivate Factors Va and VIIIa, and thus dampen the coagulation process. TPA cleaves a circulating proenzyme, plasminogen to form a plasmin, which digests fibrin nonspecifically. These down-regulation systems are obviously not available on the surfaces of mechanical devices, such as mechanical heart valves, implanted into the body, thus the surfaces of such mechanical devices promote clot formation. As used herein, “indwelling” or “implanted” means permanently or semi-permanently placed in the body, and refers to devices such as a heart valve or pacemaker.

Mechanical valve technology has struggled with the problem of valve related thrombosis and valve related thrombo-embolic events ever since the first mechanical heart valves were invented and implanted. The first heart valves had a silastic or metal ball retained inside a metal cage. While the valve worked well, catastrophic valve thrombosis was an ever-present danger. Some more recent mechanical valves no longer employ the ball valve concept but rather have a tilting bi-leaflet disk construction. Significant effort has improved more recent valve design and much study has centered around the actual mechanism of retaining the moving dual leaflets within the annulus of the valve, either by recessing or hiding the rocker mechanisms. However, virtually all patients who have a mechanical valve implanted to this day are recommended to take anti-coagulants.

Four types of medical therapies are generally available to resist the coagulation cascade from occurring: (1) antiplatelet therapy, which has not proven to be effective or safe with an implanted mechanical heart valve, (2) thrombolytic agents that induce a systemic lytic state and are neither practical nor safe for long term anti-thrombotic therapy, (3) heparin, which can be used for heart valve anti-thrombosis, but it requires daily injections and is prone to therapy errors, and (4) vitamin K antagonists (4-hydroxycoumarin, warfarin, dicumerol, indan-1.3-dione, acenocumerol and anisindione).

The use of coumadin is the current standard anticoagulant therapy for patients with an indwelling mechanical heart valve, regardless of the existing cardiac rhythm to render the blood less liable to clot on the surface of the mechanical heart valve, including the sewing ring, the valve leaflet housing and/or the leaflets themselves. The preferred anticoagulant pro-thrombin range for an aortic valve is approximately 17-19 seconds and 21-23 seconds for mitral valve patients. Thus, coumadin has a narrow therapeutic window and carries potential risks of excessive anticoagulation and thus a risk of spontaneous hemorrhage or insufficient anticoagulation with consequent catastrophic thrombo-embolism or total valve thrombosis. Due to the narrow therapeutic range and undesirable side effects of coumadin anticoagulation, considerable effort has been spent addressing this problem, but so far without success.

Further, there are occasional patients who are unknowingly intolerant of coumadin, either from an idiosyncratic allergy or a systemic intolerance or develop rare antibody resistance. These patients currently either must take other forms of anticoagulants such as self-injections of heparin daily or its derivatives or have the valve explanted and a different form of valve prosthesis must be implanted.

Even with anticoagulation, however, pannus build up on the valve annulus and/or leaflets may occur. That is usually encountered as mechanical valve re-stenosis and requires replacement of the mechanical valve.

Biological valve technology was introduced in the seventies and most biological valves do not require constant coumadin anticoagulation. The main problem with biological valves is lack of durability and most biological valves have a primary valve failure rate that becomes significant at 12-15 years after implantation.

Obviously, if a mechanical heart valve can be engineered to last for the life of the patient or longer (as measured in a pulse duplicator) it is desirable to expend considerable effort in an attempt to release the mechanical heart valve from the requirements and risks of anticoagulation.

By electrically charging an implanted mechanical device, either positively or negatively, and outside the ranges reported above, the electrostatic foreign surface attraction between the platelet and red blood cell will be altered and the intrinsic coagulation cascade will be suppressed. Such an electrified valve may require no anticoagulants, or at least fewer than are presently required.

### SUMMARY OF THE INVENTION

The present invention improves upon the prior art by providing a mechanical device that is implantable in the body and that is configured to be electrically charged by a power source. The preferred device is a mechanical heart valve and the preferred power source is a battery pack of the type that is used in pacemakers. Among the pacemaker designs that could potentially be used are those disclosed in U.S. Pat. Nos. 6,708,063, 6,505,070 and 4,201,219, the respective disclosures of which are incorporated herein by reference.

In the most preferred embodiment, the power source is attached to a heart valve by wires capable of transferring an electrical current from the power source to the device. The power source is preferably placed in a subcutaneous pocket for easy access when and if battery changes are required. The power source can supply a sufficient current to the mechanical device to sufficiently charge the device (or part of the device) to reduce or eliminate blood clotting on one or more surfaces of the device. Preferably, the power supply creates a substantially constant appropriate and substantially unipolar electrically negative (or positive) charge to the device. The electrical charge applied to the device is sufficient to repel activated platelets and activated red blood cells from settling on the charged component of the device but will be insufficient to interfere with the heart's normal beating.

The new system is expected to provide one or more of the following benefits: First, energizing an implanted mechanical device may free that device from lifelong anticoagulation requirements. Second, disclosed herein is a new form of a power source that will be capable of supplying a preferably constant electrical charge to an implanted mechanical device. Third, the power source may have a primary and secondary (redundant) source of energy, such as a first battery and a second battery, wherein the second battery supplies power if the first battery fails. Fourth, only a relatively minor modification to an existing heart valve is required so as to connect it to a power source according to the invention. In a preferred method, paired leads are attached to the valve annulus and exit either a cardiac chamber or a blood vessel to connect to a power source according to the invention. The power source is preferably implanted in a subcutaneous position in the body and can be accessed for both telemetry and changing on an as necessary basis.

### BRIEF DESCRIPTION OF THE DRAWING

FIG. 1 depicts a mechanical heart valve prostheses connected to a power source.

### DETAILED DESCRIPTION OF THE PREFERRED EMBODIMENTS

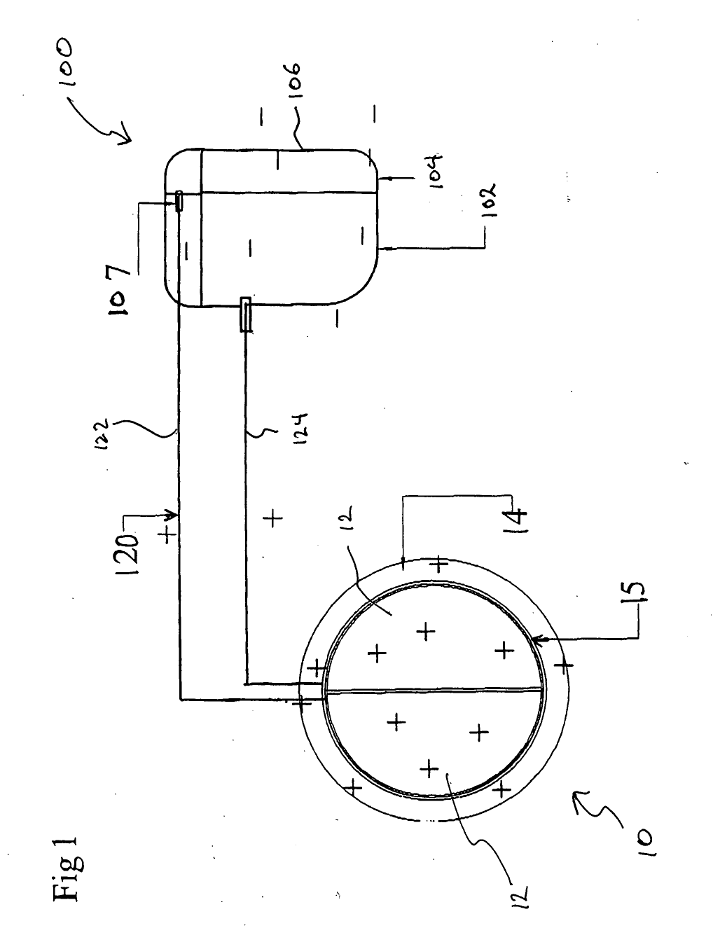

Turning now to the Drawing, where the purpose is to describe a preferred embodiment of the invention and not limit same, FIG. 1 is a schematic representation of a mechanical device and power supply according to the invention.

A device 10 according to the invention may be any mechanical device that is implanted into the body and that is susceptible to blood clotting on one or more of its surfaces to such a degree that interventional therapy is recommended to reduce or eliminate the clotting. Device 10 is preferably a heart valve, such as an aortic, tricuspid or mitral valve. Other examples of mechanical devices that may be used to practice the invention are pulmonary valves. In this embodiment, device 10 has a connective portion 11 (for receiving a connection to a power source or otherwise connecting device 10 to a power source), valve plates 12 and sewing ring 14. Device 10 can be made of any suitable materials that can be charged to prevent or alleviate blood clotting.

To render device 10 non-thrombogenic, all or part of device 10 is electrically charged, either positively or negatively, by connecting device 10 to a power source 100 that generates electrical current to charge device 10. Power source 100 is any device or system capable of electrically charging device 10 (or any part of device 10) sufficiently to alleviate or eliminate blood clotting on all or some of the surfaces of device 10. Power supply 100 is preferably a battery pack of a type already known and used with pacemakers. Power supply 100 is preferably implanted into the body in a subcutaneous pocket.

Device 10 is connected to power source 100 via a connection system 120, which is preferably a pair of wires 122, 124, and thus power source 100 electrically charges device 10. In the preferred embodiment, insulated wires 122, 124 are attached to the body of the heart valve annulus (not shown) and are then transferred out of the heart via, either the left atrium in the case of a mechanical mitral valve, the aorta in the case in a mechanical aortic valve, the right atrium in the case of a mechanical tricuspid valve implant, or the pulmonary artery in the case of a mechanical pulmonary valve, and into the pericardial space. Via the pericardial space wires 122, 124 are then brought over or under the clavical and are attached to power source 100, which is preferably a battery pack.

One difference between the functioning of power source 100 as compared to a pacemaker is that a mechanical device according to the invention should constantly be charged to prevent clotting. Since power source 100 generates the charge it may deliver power continuously to device 10 to maintain the constant charge. So, instead of providing intermittent burst current and EKG tracking and sensing capabilities as a normal pacemaker does to stimulate a heart beat when attached to the myocardium, power source 100 preferably provides a constant current via the wires and apply that current to mechanical device 10. When power source 100 is connected to a mechanical device 10, such as a heart valve, device 10 will be rendered either positively or negatively charged with respect to the blood stream, and will electrically repel activated platelets and red blood cells thus making anticoagulants unnecessary.

Preferred power source 100 is a constant discharge pacemaker-style battery pack that includes two electrically separate battery compartments 102, 104 and a casing, or cannister, 106. Cannister 106 should be laser welded and made to the same general specifications as pacemaker battery casings. The patient's body, via the power source canister will preferably act as a ground for power source 100. Each battery (not shown) preferably is capable of lasting for the patient's life. A first of the two batteries used in the preferred embodiment generates a current that charges the mechanical device and a second of the two batteries (if two batteries are used) automatically activates and generates a current that charges the mechanical device should the first battery fail or become exhausted.

Power source 100 is preferably capable of adjusting the charge output with a Battery Systems Analyzer (BSA). A hyper-dense lithium iodide battery with up to eleven years of battery life or greater is preferred as a battery to be used in power source 100. A kinetic energy recharging capability may be highly beneficial to increase battery life. Power source 100 should be capable of supplying a current anywhere between about ±100 mA and ±300 mA to mechanical device 10. Power source 100 preferably has an anode and cathode component to complete the circuit and a connector system 120 that allows leads 107 from the mechanical device to be attached to power source 100. Power source 100 and connector system 120 need to be impervious to body fluids and current pacemaker technology suffices for this purpose.

Typically, connector system 120 (in a standard pacemaker design) is housed within a cylinder of silicone through which the connector wire pin is passed. The connector wire pin then is pressed into a metal coupling. The metal coupling has a screw accessed via the silicone covering with either a small Phillips or a regular, bayonet-style screwdriver. Once the valve wire is pressed into the housing the screw is tightened and the fitting is impervious to body fluids so that corrosion and current leakage will not occur.

The first battery (not shown) should be interconnected with the (redundant) battery (not shown). The connection should have life-of-battery sensing capabilities, which would automatically activate and use the second battery when, for example, telemetrically 10% or less of first battery life is sensed. If at any time the second battery is activated the first battery should preferably be changed to insure that there is back up to maintain a charge on device 10. The second battery should have, for example, between 1 and 2 years of battery life, although any suitable life for a redundant battery is sufficient. The second battery should also have some telemetry capability. Any time the second battery is activated the first battery should be replaced.

The wires 122, 124 should be thin, and perhaps thinner than those used in current pacemakers. If the wires are too thick, they could pose bleeding problems, for example, if they exited a cardiac vascular structure. The wires will be surgically implanted and do not need steering capabilities, thus, they do not need to be thick for that purpose. Wires 122, 124 should be permanently insulated from their resting external environment. It is estimated that the wires would be supplied as part of mechanical device 10 and would thus not require any additional connection other than connection to power source 100.

Preferred mechanical device 10 is a heart valve, as previously described. Current heart valves are usually made of pyrolytic carbon, which is generally a good electrical conductor, while the sewing ring is usually made of TEFLON. Both exist in a wet (blood), turbulent, environment and will be able to accept and maintain an electrical charge. Furthermore, existing heart valves could be modified to accept an electrical charge in a manner according to the invention. Valve doors 12 may be identical to those in known valves, and the sewing annulus 14 is identical to known sewing annuluses. The only modification required is the connection for the two flexible, electrically insulated (preferably plastic coated) wires 122, 124, which would be connected to mechanical device 10. If device 10 is a heart valve, the wires would preferably be connected to the valve annulus and exit from ring 15 of the valve annulus. In the preferred embodiment, the wires would need to be long enough to traverse the cardiac structure, the pericardial space and over or under the clavical and then descend down the anterior chest wall to be pressed into the receptors of power source 100. The wire exiting from the valve could have breakable, equivalent to about a 4-0 needle thickness, 1 cm curved, round, needles (not shown) on their tips.

In use, a valve according to the invention is implanted in the heart in the normal fashion. The needles on the wires are then passed outside the heart. Once they are ready to be attached to the power source (and thus preferably electrically connected to the first battery and second battery) the needles are snapped off and the stump of the needles are inserted into the power source housing and are screw tightened to be retained.

Standard trial and error, done using techniques known to those skilled in the art, will indicate the necessary charge to repel platelets and passing red blood cells, but in general the current necessary can be expected to lie somewhere between ±100 to 300 milliamps and/or a charge of ±100 to 300 millivolts must be applied to device 10. It might need to be higher than that charge depending upon the indexed mass of the individual. In the event that a multiple heart valve implantation is made all valves could be charged utilizing the invention.

Having now described preferred embodiments of the invention, modifications and variations to the present invention may be made by those skilled in the art. The invention is thus not limited to the preferred embodiments, but is instead set forth in the following claims and legal equivalents thereof.

1. A mechanical device for implantation into a body, the device configured to be connectable to a power source for electrically charging the device and thereby lessening coagulation at lest part of the surface of the device by repelling at least some platelets and red blood cells.

2. The mechanical device of claim 1 wherein the device is a heart valve.

3. The device of claim 1 wherein the device is a pulmonary valve.

4. The device of claim 2 wherein the device is a tricuspid valve.

5. The mechanical device of claim 2 wherein the device is a mitral valve.

6. The mechanical device of claim 2 wherein the device is an aortic valve.

7. The device of claim 1 that is connected to a power source, wherein the power source is capable of applying an electrical charge to the device.

8. The device of claim 1 that is electrically charged.

9. The device of claim 8 that is constantly electrically charged.

10. The device of claim 1 wherein an electric current is constantly supplied to the device by the power source.

11. The device of claim 7 that is connected to the power source by one or more wires that can transfer electric current from the power source to the device.

12. The device of claim 11 wherein the power source is a battery pack.

13. The device of claim 7 wherein the power source is a battery pack.

14. The device of claim 13 wherein the battery pack has two batteries.

15. The device of claim 13 wherein the battery pack comprises a canister that retains the batteries therein.

16. The device of claim 15 wherein the canister functions as a ground for electrical current generated by the power source.

17. The device of claim 7 wherein the power source generates a negative charge in the device.

18. The device of claim 7 wherein the power source generates a positive charge in the device.

19. The device of claim 14 wherein each of the batteries is electrically isolated from the other.

20. The device of claim 7 wherein the power source is subcutaneously implanted.

21. The device of claim 14 wherein there is a first battery and a second battery, and at least one wire connects the first battery to the device and at least one wire connects the second battery to the device, wherein the at least one wire that connects the first battery to the device is a different wire than the at least one wire that connects the second battery to the device.

22. The device of claim 14 wherein a first pair of wires connects the first battery to the device and a second pair of wires connects the second battery to the device.

23. The device of claim 11 wherein the one or more wires are connected to the body of the valve annulus.

24. The device of claim 11 wherein the one or more wires are insulated.

25. The device of claim 11 wherein the device is a heart valve and the one or more wires are connected to the heart valve and pass through the left atrium in the case of a mitral valve, the aorta in the case of an aortic valve, and the right atrium in the case of a tricuspid valve, into the pericardial space, over the clavical and are connected to the power source.

26. The device of claim 13 wherein the battery pack includes a lithium iodide battery.

27. The device of claim 2 wherein the heart valve comprises pyrolytic carbon.

28. The device of claim 2 wherein the heart valve has a sewing ring, the sewing ring comprising TEFLON.

29. The device of claim 7 wherein the power source is designed to last for the life of the patient.

30. The device of claim 14 wherein there is a first battery and a second battery, and the first battery supplies power to the device until it is incapable of doing so, at which time the second battery supplies power to the device.

31. The device of claim 7 wherein the power source generates a voltage of between 100 mV and 300 mV.

32. The device of claim 7 wherein the power source generates a current of between 100 mA and 300 mA.

33. A power source for implantation in a body, wherein the power source is connectable to a mechanical device implanted in the body to electrically charge the device by applying an electrical current to the device.

34. The power source of claim 33 that supplies a constant current to the device.

35. The power source of claim 33 that is a battery pack.

36. The power source of claim 33 that comprises two electrically isolated batteries, wherein a first of the two batteries generates an electrical charge in the device and second of the two batteries generates an electrical charge to the device should the first battery malfunction or become exhausted.

37. The power source of claim 36 wherein if the second of the two batteries, is activated, mandates that the first of the two batteries be replaced.

38. The power source of claim 33 that includes a pair of insulated connector wires that connect the power source to the device in a manner that prevents body fluids from entering the power source.

39. The power source of claim 33 wherein the power source is a battery pack.

40. The power source of claim 39 wherein the battery pack has two batteries.

41. The power source of claim 39 wherein the battery pack comprises a canister that retains the batteries therein.

42. The power source of claim 41 wherein the canister functions as a ground for electrical current generated by the power source.

43. The power source of claim 33 that generates a negative charge in the device.

44. The power source of claim 33 that generates a positive charge in the device.

45. The power source of claim 33 that is subcutaneously implanted.

46. The power source of claim 40 wherein there is a first battery and a second battery, and at least one wire connects the first battery to the device and at least one wire connects the second battery to the device, wherein the at least one wire that connects the first battery to the device is a different wire than the at least one wire that connects the second battery to the device.

47. The power source of claim 46 wherein a first pair of wires connects the first battery to the device and a second pair of wires connects the second battery to the device.

48. A method for rendering an existing hart valve partially or entirely non-thrombogenic by attaching a pair of insulated wires to the annulus of the heart valve, wherein the wires exit the heart to connect to a power source.

49. The method of claim 48 wherein the power source is a battery pack.

50. The method of claim 48 wherein the wires exit the left atrium of the heart in the case of a mitral valve or the aorta in the case of an aortic valve and reach the pericardial space.

51. The method of claim 48 wherein the wires are of a small diameter so as to reduce the likelihood of post operative bleeding after insertion.

52. The method of claim 48 wherein the electrical connection between the power source and the wires are made outside the heart.

53. The method of claim 48 wherein the power source generates a charge to be applied to the heart valve annulus, the body of the annulus and the valve leaflets.

54. The method of claim 48 wherein the power source is capable of supplying sufficient current to electrically charge the annulus and the entire valve structure of a heart valve.
